(19) United States
(12) Patent Application Publication
(10) Pub. No.: US 2025/0292456 A1
(43) Pub. Date: Sep. 18, 2025

(54) ENERGY-RESOLVED IMAGE RECONSTRUCTION

(71) Applicant: Siemens Medical Solutions USA, Inc., Malvern, PA (US)

(72) Inventor: Peter Adany, Hoffman Estates, IL (US)

(21) Appl. No.: 18/607,823

(22) Filed: Mar. 18, 2024

(51) Int. Cl. G06T 11/00; A61B 6/03; A61B 6/42

(52) U.S. Cl. CPC G06T 11/005; A61B 6/037; A61B 6/4241

(57) ABSTRACT

A framework for energy-resolved image reconstruction. The framework receives projection data representing emissions detected from a subject. The projection data may be formatted into energy-resolved data. Contribution coefficients of one or more components of the emissions may be determined based on the energy-resolved data. An image of the subject may be reconstructed using the contribution coefficients.

### TECHNICAL FIELD

The present disclosure generally relates to data processing, and more particularly to a framework for energy-resolved image reconstruction.

### BACKGROUND

The imaging of radionuclides emitting negatively charged beta particles (β⁻) using single photon emission computed tomography (SPECT) imaging is desirable for cancer treatment assessment (i.e., “theranostics”). In such treatment, microspheres containing a radioactive isotope such as Yttrium-90 (Y-90), Holmium-166, or others are delivered arterially to cancerous tissue where they embolize the mass. Radioactive decay of the atomic nuclei emits energetic β⁻ particles that damage the nearby cancer cells. See, for example, d'Abadie et al., Microspheres Used in Liver Radioembolization: From Conception to Clinical Effects, Molecules 2021, 26, 3966, which is herein incorporated by reference.

Deceleration of β⁻ particles by interactions with the surrounding matter results in emission of “bremsstrahlung radiation,” having a broad and continuous energy distribution. It is desirable to image bremsstrahlung radiation to assess dose and targeting efficacy in Y-90 radioembolization treatment. The β⁻ particle has a penetrating range of only a few millimeters in soft tissues, whereas the generated bremsstrahlung photons can penetrate soft tissues and reach the detector. Imaging of such emissions is challenging for SPECT systems, which are primarily designed for use with γ (“gamma”) emitters. γ decays have distinct emission energies that can be detected by the energy-discriminating SPECT gamma camera as “photopeaks.” In bremsstrahlung imaging, relatively few direct (un-scattered) photons are detected. Therefore, an improved method of SPECT (and/or planar) imaging of bremsstrahlung emissions from Y-90 isotope is desirable to evaluate its spatial distribution in the anatomy for radioembolization treatment.

Because detected bremsstrahlung photons have a continuous, broad and complicated energy spectrum with relatively low yield of direct photons, unlike other radioisotopes used for diagnostic imaging, Y-90 SPECT images usually have poor contrast. Photon interactions with the detector apparatus create unwanted contributions from camera backscatter, collimator scatter and septal penetration, as well as lead fluorescence X-rays. Traditional energy-windowed acquisition has been insufficient to remove the unwanted contributions due to spectral overlap of the contributions.

### SUMMARY

Described herein is a framework for energy-resolved image reconstruction. The framework receives projection data representing emissions detected from a subject. The projection data may be formatted into energy-resolved data. Contribution coefficients of one or more components of the emissions may be determined based on the energy-resolved data. An image of the subject may be reconstructed using the contribution coefficients.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 is a block diagram illustrating an exemplary system;

FIG. 2 shows an exemplary method of image reconstruction;

FIG. 3 shows exemplary results of non-negative least squares regression fitting;

FIG. 4 shows exemplary reconstructed images of different detected emission components;

FIG. 5 shows exemplary original and reconstructed images of a subject; and

FIG. 6 shows an exemplary image of uncorrected framed photopeak data and an exemplary image of corresponding corrected high resolution framed photopeak data.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance. Independent of the grammatical term usage, individuals with male, female or other gender identities are included within the term.”

Unless stated otherwise as apparent from the following discussion, it will be appreciated that terms such as “segmenting,” “generating,” “registering,” “determining,” “aligning,” “positioning,” “processing,” “computing,” “selecting,” “estimating,” “detecting,” “tracking” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, implementations of the present framework are not described with reference to any particular programming language. It will be appreciated that a variety of programming languages may be used.

The characteristics of spectral components detected by gamma (or SPECT) cameras have previously been investigated with regards to bremsstrahlung SPECT imaging. See, for example, Walrand et al., Optimal design of Anger camera for bremsstrahlung imaging: Monte Carlo evaluation, Front. Oncol. 2014, 4, 149; and Heard et al., Monte Carlo Simulation of ⁹⁰Y Bremsstrahlung Imaging, IEEE Nucl. Sci. Symp. 2004, 7, 3579-83, which are herein incorporated by reference. It has been shown that various categories of energy spectral contributions in SPECT imaging of bremsstrahlung emissions may be estimated using the techniques of Monte Carlo simulation for various SPECT systems. Such works characterized the individual contributions with the major gamma camera components. Other approaches to estimate the emission spectra in physical systems may also include analytic calculations, machine learning algorithms, and other methods.

Some approaches have simulated energy spectral contributions in Y-90 SPECT, and they have incorporated them as spectral models in iterative reconstruction. Some methods used fewer than ten energy windows due in part to limitations imposed by the computational demands. Subdividing emissions data into a small number of energy windows, i.e. fewer than ten, usually will not provide enough differentiation to separate signals based solely on their energy spectral patterns. These methods may rely on absolute accuracy and realism of the physical model to estimate the undesired contributions, which is impractical to achieve in clinical imaging scenarios. Therefore, such methods may incorporate unforeseen errors.

The present framework extends the concept of energy-resolved processing towards a computationally efficient means of image reconstruction and/or correction. In some implementations, the input data is first formatted in such a way that a linear algebraic reconstruction may be performed over the energies belonging to each spatial position. Methods including, but not limited to, non-negative constrained regularized least squares regression (LSR), Maximum Likelihood Expectation Maximization (MLEM), and Conjugate Gradient (CG) method, may then be performed with the system matrix containing the basis energy spectra of various contributions to isolate subject emission components (e.g., primary, scatter, lead X-rays, camera backscatter, collimator scatter and penetration), yielding contribution coefficients of the emissions and unwanted background signals. The subject and background images may be reconstructed from these coefficients separately, and image correction may be performed using the reconstructed coefficients.

The present framework may be more effective in separating energy spectral contributions. It may also be more computationally efficient than methods that extend iterative spatial reconstruction in the energy dimension. Additionally, the present framework may reduce reconstruction errors caused by unknown scaling factors in physical modeling. It may also be useful in non-tomographic applications (e.g., planar imaging). Significant reduction of non-subject scatter may be achieved. With improved data and model consistency, this framework may also be less sensitive to modeling inaccuracies. These and other exemplary advantages and features will be described in more detail in the following description.

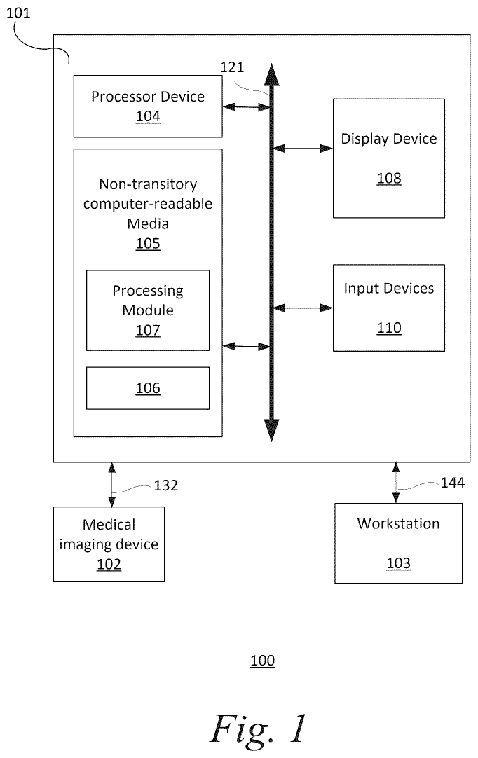

FIG. 1 is a block diagram illustrating an exemplary system 100. System 100 includes a computer system 101 for implementing the framework as described herein. In some implementations, computer system 101 operates as a standalone device. In other implementations, computer system 101 may be connected (e.g., using a network) to other machines, such as medical imaging device 102 and workstation 103. In a network implementation, computer system 101 may operate in the capacity of a server or a client in a server-client user network environment, or as a peer machine in a peer-to-peer (or distributed) network environment).

In one implementation, computer system 101 includes a processor device or central processing unit (CPU) 104 coupled to one or more non-transitory computer-readable media 105 (e.g., computer storage or memory device), display device 108 (e.g., monitor) and various input devices 110 (e.g., mouse, touchpad or keyboard) via an input-output interface 121. Computer system 101 may further include support circuits such as a cache, a power supply, clock circuits, and a communications bus. Various other peripheral devices, such as additional data storage devices and printing devices, may also be connected to the computer system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in one or more non-transitory computer-readable media 105. In particular, the present techniques may be implemented by a processing module 107. Non-transitory computer-readable media 105 may include random access memory (RAM), read-only memory (ROM), magnetic floppy disk, flash memory, and other types of memories, or a combination thereof. The computer-readable program code is executed by processor device 104 to process data provided by, for example, medical imaging device 102 and database 106. As such, computer system 101 is a general-purpose computer system that becomes a specific-purpose computer system when executing the computer-readable program code. The computer-readable program code is not intended to be limited to any particular programming language and implementation thereof. It will be appreciated that a variety of programming languages and coding thereof may be used to implement the teachings of the disclosure contained herein. The same or different computer-readable media 105 may be used for storing a database 106, including, but not limited to, image datasets, a knowledge base, individual subject data, electronic medical records (EMRs), diagnostic reports (or documents) for subjects, or a combination thereof.

Medical imaging device 102 acquires medical image data 132. Such medical image data 132 may be processed by processing module 107. Medical imaging device 102 may be a radiology scanner and/or appropriate peripherals (e.g., keyboard and display device) for acquiring, collecting and/or storing such medical image data 132. In some implementations, medical imaging device 102 performs medical imaging with multiple emission energies. Medical imaging device 102 may acquire medical image data 132 from a subject or patient by using techniques such as planar imaging or tomographic imaging such as single photon emission computed tomography (SPECT). Other types of imaging modalities are also useful. Medical image data 132 is data that represents a medical image (or in certain examples more than one medical image). For example, medical image data 132 may comprise projection data representing emissions from the subject. When processed by suitable image viewing software, the medical image data 132 results in a rendering of the medical image (or medical images) that it represents.

Workstation 103 may include a computer and appropriate peripherals, such as a keyboard and display device, and can be operated in conjunction with the entire system 100. For example, workstation 103 may communicate with medical imaging device 102 so that the medical image data 132 can be presented or displayed at the workstation 103. The workstation 103 may communicate directly with the computer system 101 to display processed data and/or output results 144. The workstation 103 may include a graphical user interface to receive user input via an input device (e.g., keyboard, mouse, touch screen, voice or video recognition interface, etc.) to manipulate visualization and/or processing of the data.

It is to be further understood that, because some of the constituent system components and method steps depicted in the accompanying figures can be implemented in software, the actual connections between the systems components (or the process steps) may differ depending upon the manner in which the present framework is programmed. Given the teachings provided herein, one of ordinary skill in the related art will be able to contemplate these and similar implementations or configurations of the present framework.

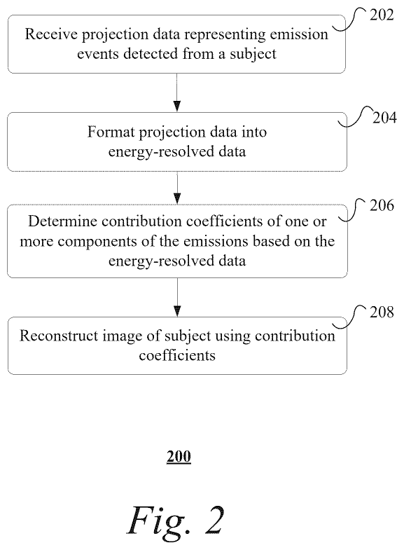

FIG. 2 shows an exemplary method 200 of image reconstruction. It should be understood that the steps of the method 200 may be performed in the order shown or a different order. Additional, different, or fewer steps may also be provided. Further, the method 200 may be implemented with the system 100 of FIG. 1, a different system, or a combination thereof.

At 202, processing module 107 receives projection data of emissions or emission events detected from imaging acquisition of a subject (or, object) in which one or more radionuclides have been introduced (e.g., ingested or injected). Projection data may represent detected positions and detected energies of emission events. The emissions may have a continuous energy spectrum or distinct energy peaks. A continuous energy spectrum may be emitted by, for example, bremsstrahlung radiation resulting from Y-90 decay. Discrete energy peaks may be emitted from, for example, Lu-177. Other types of emissions are also possible.

Medical imaging device 102 may include a single-photon emission computed tomography (SPECT) camera that is positioned relative to the subject to acquire the projection data. The SPECT camera performs SPECT imaging, which involves measuring photon radiation in the gamma energy range to map out their spatial origins in the patient. The SPECT camera may be used for either “tomographic” or single “planar” imaging. In some implementations, the SPECT camera includes Thallium-doped Sodium Iodide NaI(TI) scintillating crystal, light-coupling media, photomultiplier (PMT), and back-end electronics. A shielded enclosure may house the scintillating crystal and detector electronics. A collimator may be positioned in front of the camera to limit the direction of emissions detected by the camera, so that each detected emission is associated with an energy and line or cone of possible locations from which the emission occurred. Emissions from the one or more radionuclides (e.g., Y-90) are detected from multiple view angles for tomographic imaging. A sinogram image of accumulated emission counts at different view angles and radial distances may be formed.

The energies, positions, times and/or orders of arrival of emissions from the one or more radionuclides may be measured by the SPECT camera of the medical imaging device 102 and stored as “list mode data.” A dataset of list mode data may continue to accumulate with processing being initiated before the end of acquisition. Medical imaging device 102 may record the energy and position of detected photons as projection data. Energy values may be recorded with a finite precision numeric representation of, for example, integer or floating-point format. The stored values may be translated to physical units such as keV by linear or non-linear mapping.

A small portion of detected counts are “primary” photons that traverse a line from their point of origin to the scintillating crystal through a collimator hole. Another portion of counts are photons scattered within the subject (“subject scatter”) that similarly pass cleanly through the collimator. Additionally, there are nuisance contributions that lack useful spatial information: some of the photons penetrate the collimator septa where they may scatter or they may be absorbed and cause re-emission as X-Ray fluorescence; and, they may penetrate through the crystal and cause backscatter from the detector assembly behind the crystal. Although the Y-90 β⁻ transition energy of 2.3 MeV may be above the detector's energy range, some high-energy photons may “scatter down” into the detector's energy range.

As described, emissions detected by the detector may be a combination of different emission components: (1) primary photons (desired un-scattered photons) (“primary component”), (2) photons scattered inside body of the subject (“subject scatter component”), (3) photons penetrating a collimator septa (“penetration component”), (4) photons scattered by a collimator septa (“collimator scatter component”), (5) lead X-ray emission photons (“lead X-ray component”), and (6) back-scatter photons from the camera (“camera backscatter component”). Components (2) through (6) may be considered to be image-degrading components that are undesired. However, in practice, components (3) through (6) may usually be removed from the resulting image.

At 204, processing module 107 formats the projection data into energy-resolved data. Processing module 107 may format the projection data into “framed” images of each angular “view”. Multiple frames may be combined, for example, to mitigate attenuation effects by combining frames of two detectors facing 180 degrees to each other. The data formatting (“framing”) makes use of an energy inclusion range criteria. Traditionally, the included energies span relatively wide, factory-defined or user pre-selected ranges (or “windows”), and such ranges are associated with particular major peaks and/or minor peaks. An often-used image correction method is “scatter correction,” in which the image framed with a scatter window is scaled and subtracted from the primary image framed with a photopeak window. Such correction is a simple method for discriminating signals based on information in the energy spectra.

For the present framework, data may be framed with multiple narrow (e.g., 5 keV) energy windows. Keeping the physical parameters of acquisition fixed, a narrower energy window results in less count density in the framed images. To overcome the variance of the Poisson process associated with nuclear decays, a relatively small framed image size (e.g., 64 by 64 pixels) may be used. A choice of low-resolution framing may risk removing some spatial information, but it can often be chosen such that the spatial bandwidth is above that of the features of interest. An advantage of lower-resolution framing is the improved ability to evaluate image content of one angle of view independently of others.

In some implementations, each spatial point (or pixel) in the framed view is treated independently, wherein the coefficients for that point (or pixel) may be determined by linear algebraic methods. To attain sufficient energy definition, the projection data may be formatted into many frames corresponding to relatively narrow energy windows over a wide energy band, with each frame having a relatively small image size. Fewer and wider energy windows result in less differentiation of their spectral shapes, while more and narrower energy windows result in higher differentiation. In some implementations, the projection data is formatted into 20 or more non-overlapping energy windows. For example, the projection data may be formatted into 40 frames over an energy band of 40-240 keV, with each frame corresponding to a relatively narrow energy window of 5 keV and represented by a relatively small image size of 64 by 64 pixels. As another example, the projection data may be formatted into 110 frames over an energy band of 40-590 keV, with each frame corresponding to a relatively narrow energy window of 5 keV and represented by a relatively small image size of 64 by 64 pixels. Other formats are also useful.

At 206, processing module 107 determines the contribution coefficients of one or more components of the emissions based on the energy-resolved data. As discussed previously, the emissions detected by the detector may include different components (e.g., primary, subject scatter, lead X-rays, camera backscatter, collimator scatter, penetration components). These emission components may be isolated using knowledge about the various spectral contributions.

Contributions to an acquired energy spectrum may be considered as a linear system,

$$s = Hc + n \qquad ( 1 )$$

where s∈ [symbol] ^{M} is a vector of the counts data derived from the energy-resolved data, H∈ [symbol] ^{M-N} is a matrix with M rows and N columns, c∈ [symbol] ^{N} is a vector of the contribution coefficients, and n∈ [symbol] ^{M} represents additive noise. Vector s may be extracted from the formatted projection data.

In some implementations, H is a matrix that contains a model for various contributions to the measured data, subject to a scaling factor, with each contribution contained as a column of the matrix. Matrix H may be predetermined using modeling techniques, such as simulation or artificial intelligence methods. It will be known to those skilled in the art that such a linear system of equations allows exact or approximate coefficients, ĉ, to be found given some data, s, using linear algebraic techniques. Generally, the matrix (H) need not be a square matrix: if M>N, the system is overdetermined and ĉ may be found using least squares methods.

The physical system does not allow for negative contributions. Therefore, the estimation of coefficients given input data, s derived from the energy-resolved data and initial assumptions or prior information may be posed as a non-negative regularized constrained least squares problem:

$$\hat{c} = arg {min}_{c} ‖ s - Hc ‖ + ‖ Wc ‖ subject to c \geq 0 \qquad ( 2 )$$

where W is a regularization matrix, and usually W=αI where I is the identity matrix and α is a weight on the regularization. The present framework performs such estimation and obtains the coefficients, ĉ.

The present framework observes that the energy spectra of the described contributions are usually linearly independent. Therefore, the individual contributions comprise a basis for a subspace in R^{M}. Detected energy spectral data from bremsstrahlung emissions may be considered as residing in this subspace: the information contained in the detected spectral shapes may be sufficient to allow separation into the subject (e.g., primary and scatter) emissions and nuisance contributions (e.g., lead X-Rays, collimator scatter and penetration, camera backscatter). The amount of each contribution may be non-negative, but otherwise unknown due to additional scaling factors in detection. Non-negative constrained least squares regression may be applied to solve for the various contributions in the continuous energy sinograms.

Furthermore, the system may be described by considering the Poisson statistics of the emission process, as follows:

$$E \{ s \} = G λ \qquad ( 3 )$$

where G is a transition probability matrix, E{s} is the expectation of the observed counts derived from the energy-resolved data, and A are coefficients identified as the emission Poisson parameters for the contributions. A Poisson-distributed random variable has the probability distribution,

$$P ( k , λ ) = \frac{{e}^{- λ} {λ}^{k}}{k !} ,$$

k=0,1,2, . . . , wherein parameter λ is the expected mean number of events in a given time period, and k is the number of events observed. G represents the probabilities, hence G is the H matrix normalized such that the sum of each column is 1. The system of Equation 3 may be solved by applying, for example, the technique of Maximum-Likelihood Expectation-Maximization Algorithm (MLEM). See, for example, Shepp L A, Vardi Y, Maximum likelihood reconstruction for emission tomography, IEEE Trans Med Imaging. 1982;1(2):113-22, which is herein incorporated by reference. Other methods, such as conjugate gradient (CG) methods, are also useful. Hence, the present framework solves the problem of separating emission contributions by presuming that the energy spectra of the contributions may be modeled as a linear system using a suitable energy-spectral basis.

The energy spectra of each emission contribution type may be modeled. In some implementations, a scatter model is used. A model-based scatter estimation may be provided by modeling the physics of scatter in the subject and surrounding matter. A physically-based simulation, such as the Monte Carlo method or the quasi-Monte Carlo method, may be used to model the scatter. The scatter may be modeled differently for different energies. Photons with different energies may scatter differently.

Linear algebraic analysis may be applied to each energy spectrum to model the relationship between the contribution coefficient c to the matrix H. In some implementations, non-negative least squares regression (LSR) analysis is performed on the energy spectra to yield the various contribution coefficients. The problem of non-negative LSR is a type of constrained least square problem where the coefficients are not allowed to become negative. Other types of linear algebraic techniques may also be performed, which model the relationship between the emission Poisson parameters λ to the transition probability matrix G. In those implementations, iterative reconstruction (e.g., MLEM, CG) is performed to estimate the emission Poisson parameters. In some implementations, processing module 107 applies algebraic reconstruction to four-dimensional (4D) formatted data representing the detected emission activity with two-dimensional detector spatial coordinates, the angle of view dimension, and the energy dimension, by treating the image pixels of each angle of view as independent of the others.

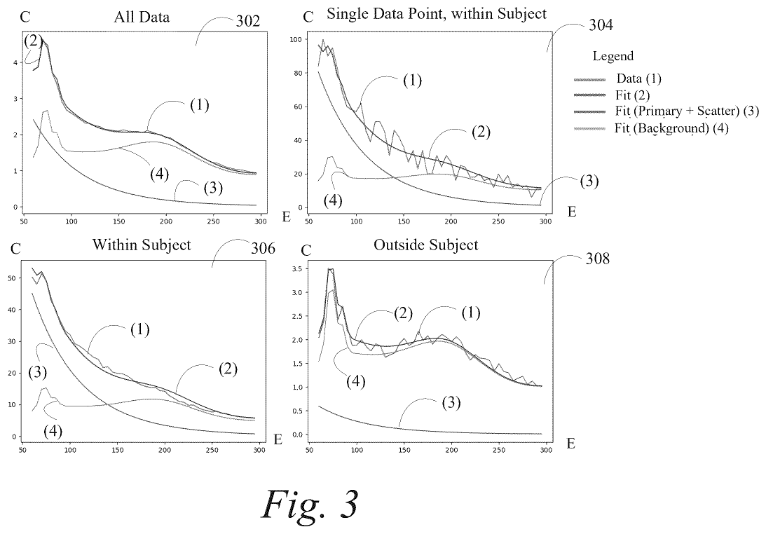

FIG. 3 shows exemplary results of non-negative LSR fitting. Chart 302 shows the non-negative LSR fitted lines of all the data, including the primary and scatter components as well as the unwanted background components. The vertical axis represents the counts (C) in arbitrary units, and the horizontal axis represents energy (E) in keV. Primary and scatter components include detected primary photons (desired un-scattered photons) and scatter photons inside the object of interest. Background components include detected X-ray emission photons, camera backscatter photons, and collimator scatter and penetration photons. Chart 304 shows the non-negative LSR fitted lines of data derived from a single data point within the subject. Chart 306 shows the non-negative LSR fitted lines of data representing counts within the subject. Chart 308 shows the non-negative LSR fitted lines of data representing counts outside the subject.

Returning to FIG. 2, at 208, processing module 107 reconstructs one or more images of the subject using the contribution coefficients. The reconstructed images of the subject represent the activity distribution in the subject, and they may be reconstructed based on the contribution coefficients of the primary and scatter components. Images of the background may be reconstructed separately based on the contribution coefficients of the remaining emission components.

The image reconstruction process may be iterative. Reconstruction includes a projection operator (i.e., forward projector) that incorporates the effects of the SPECT camera on the photons (i.e., collimation and detection process). The forward projector contains a model of the imaging formation process. The image formation model includes the interaction of photons with patients (e.g., attenuation and scatter), the collimation-detection process (e.g., collimator detector response including collimator geometric response, septal penetration, and scatter, partial deposition in crystal and detector intrinsic resolution), and related radionuclide properties (e.g., emission abundances). The image reconstruction method may include, for example, Maximum Likelihood Expectation Maximization (ML-EM), Ordered Subset Expectation Maximization (OSEM), penalized weighted least squares (PWLS), Maximum A Posteriori (MAP), multi-modal reconstruction, or other approaches. The present framework may serve as the image reconstruction, or it may serve as a correction to images subject to other image reconstruction methods.

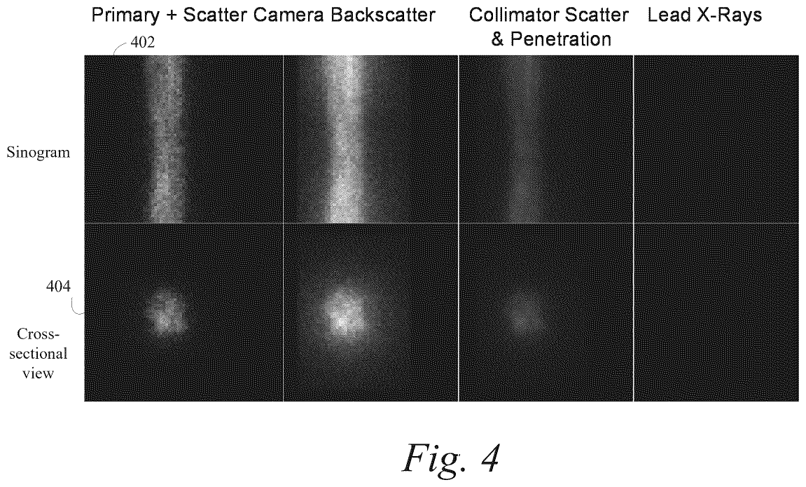

FIG. 4 shows exemplary reconstructed images (402, 404) of different detected emission components. The different detected emission components include the primary and scatter photons, the camera backscatter photons, the collimator scatter and penetration photons, and the lead X-ray photons. The top row of images 402 shows sinograms from a selected image axial slice position, while the bottom row of images 404 shows cross-sectional views for the selected slice position. It can be observed that there is good object separation and mostly non-zero coefficients.

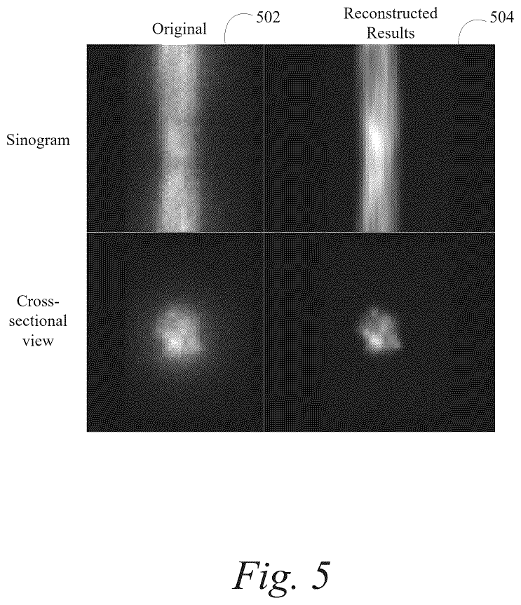

FIG. 5 shows exemplary original images 502 and the reconstructed images 504 of a subject. The upper row displays sinogram views of the data and the lower row displays cross-sectional views. The reconstructed images 504 have been reconstructed at 64×64 matrix size. A forward projection of the data demonstrates observable improvement in image quality. A significant reduction of non-subject scatter is achieved.

The foregoing methods estimate the coefficients of subject and non-subject emissions, wherein image correction may be applied to remove non-subject emissions. It may be desirable to apply such correction to framed data with arbitrary and higher resolution. For example, corrections may be applied at the increased resolution, and corrections may be applied to individual detector framed images, e.g., with 180-degree opposing views.

The low-resolution reconstruction energy-resolved coefficients ĉ may be converted into weighted scaling factors \hat{ƒ} representing the primary and subject scatter components (components (1) and (2)) as a fraction of the total counts. The low spatial resolution weighted scaling factors \hat{ƒ} may be converted to factors Fᵢⱼ of dimensions I×J, i=1, . . . , I and j=1, . . . , J with an arbitrary (usually higher) nominal spatial resolution. This may be done by, for example, performing discrete Fourier transform operations on the coefficients ĉ. Other methods, such as image interpolation methods (e.g., linear or spline interpolation), may be used. The resulting data may then be padded with zeros, and inverse discrete Fourier transform may be performed on the zero-padded data.

A set of framed data, Λᵢⱼ, i=1, . . . , I and j=1, . . . ,/represents a single observation of the counts derived from the projection data. For this single observation of Poisson distributed data, Λᵢⱼ can be understood as the maximum likelihood estimate of the Poisson parameter. In removing counts associated with non-subject emissions, Poisson statistics may be preserved to the extent possible, and it may be preferred not to disturb zero-valued elements nor to result in negative counts. These objectives may be met by reconstructing the high-resolution framed data Rᵢⱼ as follows:

$${R}_{ij} = ∼ P ( {F}_{ij} {Λ}_{ij} ) i = 1 , … , I and j = 1 , … , J \qquad ( 4 )$$

wherein ˜P (λ) denotes values drawn from a Poisson random process with parameter λ. Finally, additional filtering, smoothing, sharpening and other techniques may be used to modify and condition the high-resolution weighted scaling factors Fᵢⱼ, and they may incorporate goodness of fit, residual sum of squares, confidence intervals, Chi-squared, and other metrics to mitigate uncertainty and noise.

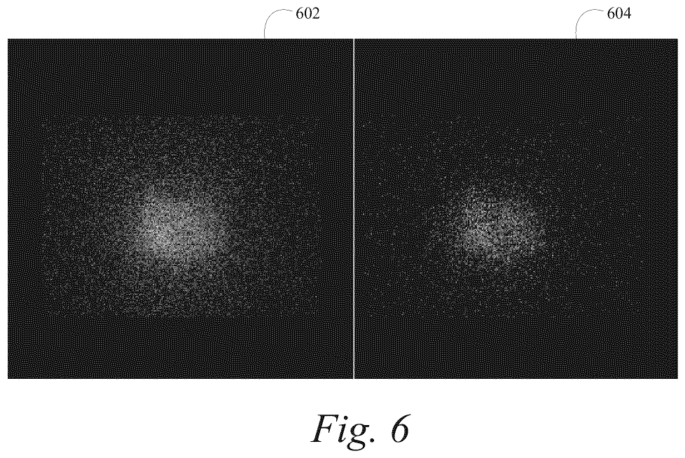

FIG. 6 shows an exemplary image 602 of uncorrected framed photopeak data Λᵢⱼ and an exemplary image 604 of corresponding corrected high-resolution framed photopeak data Rᵢⱼ. The images (602 and 604) are 256 by 256 pixels and are derived from a 90 to 125 keV energy window with intensity gamma curve at 0.5. The image 604 has been corrected using MLEM reconstruction of the coefficients. It can be observed that there are significantly reduced background counts and preserved object counts in the corrected image 604.

The following is a list of non-limiting illustrative embodiments disclosed herein:

Illustrative embodiment 1. An image reconstruction system, comprising: a non-transitory memory device for storing computer readable program code; and a processor device in communication with the non-transitory memory device, the processor device being operative with the computer readable program code to perform steps including receiving projection data representing emissions detected from a subject, formatting the projection data into energy-resolved data, determining contribution coefficients of one or more components of the emissions based on the energy-resolved data, and reconstructing an image of the subject using the contribution coefficients.

Illustrative embodiment 2. The image reconstruction system of illustrative embodiment 1 wherein the emissions have a continuous energy spectrum.

Illustrative embodiment 3. The image reconstruction system of illustrative embodiment 2 wherein the emissions are detected from bremsstrahlung radiation

Illustrative embodiment 4. The image reconstruction system of any one of illustrative embodiments 1-3 wherein the processor device is operative with the computer readable program code to format the projection data into the energy-resolved data by framing the projection data into multiple non-overlapping energy windows.

Illustrative embodiment 5. The image reconstruction system of illustrative embodiment 4 wherein at least one of the non-overlapping energy windows corresponds to an energy band of 5 keV.

Illustrative embodiment 6. The image reconstruction system of illustrative embodiment 4 wherein at least one of the non-overlapping energy windows is represented by an image size of 64 pixels by 64 pixels.

Illustrative embodiment 7. The image reconstruction system of illustrative embodiment 4 wherein the multiple non-overlapping energy windows comprises 20 or more non-overlapping energy windows.

Illustrative embodiment 8. The image reconstruction system of any one of illustrative embodiments 1-7 wherein the processor device is operative with the computer readable program code to determine the contribution coefficients of the one or more components of the emissions by performing non-negative least squares regression (LSR) analysis on the energy-resolved data.

Illustrative embodiment 9. The image reconstruction system of any one of illustrative embodiments 1-8 wherein the processor device is operative with the computer readable program code to determine the contribution coefficients of the one or more components of the emissions by performing Maximum-Likelihood Expectation-Maximization Algorithm (MLEM).

Illustrative embodiment 10. The image reconstruction system of any one of illustrative embodiments 1-9 wherein the processor device is operative with the computer readable program code to reconstruct the image of the subject based on the contribution coefficients of primary and subject scatter components.

Illustrative embodiment 11. The image reconstruction system of any one of illustrative embodiments 1-10 wherein the processor device is operative with the computer readable program code to further convert the contribution coefficients to weighted scaling factors representing primary and subject scatter components and reconstruct the image of the subject using the weighted scaling factors.

Illustrative embodiment 12. The image reconstruction system of illustrative embodiment 11 wherein the processor device is operative with the computer readable program code to convert the weighted scaling factors to higher spatial resolution weighted scaling factors.

Illustrative embodiment 13. A method, comprising: receiving projection data representing emissions detected from a subject; formatting the projection data into energy-resolved data; determining contribution coefficients of one or more components of the emissions based on the energy-resolved data; and reconstructing an image of the subject using the contribution coefficients.

Illustrative embodiment 14. The method of illustrative embodiment 13 wherein formatting the projection data into the energy-resolved data comprises framing the projection data into multiple non-overlapping energy windows.

Illustrative embodiment 15. The method of illustrative embodiment 14 wherein at least one of the non-overlapping energy windows corresponds to an energy band of 5 keV.

Illustrative embodiment 16. The method of any one of illustrative embodiment 13-15 wherein determining the contribution coefficients comprises performing non-negative least squares regression (LSR) analysis on the energy-resolved data.

Illustrative embodiment 17. The method of any one of illustrative embodiments 13-16 wherein determining the contribution coefficients comprises performing Maximum-Likelihood Expectation-Maximization Algorithm (MLEM).

Illustrative embodiment 18. The method of any one of illustrative embodiments 13-17 wherein determining the contribution coefficients comprises performing a conjugate gradient method.

Illustrative embodiment 19. The method of any one of illustrative embodiments 13-18 further comprises converting the contribution coefficients to weighted scaling factors representing primary and subject scatter components and reconstructing the image of the subject using the weighted scaling factors.

Illustrative embodiment 20. One or more non-transitory computer readable media embodying a program of instructions executable by machine to perform steps comprising: receiving projection data representing emissions detected from a subject; formatting the projection data into energy-resolved data; determining contribution coefficients of one or more components of the emissions based on the energy-resolved data; and reconstructing an image of the subject using the contribution coefficients.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. An image reconstruction system, comprising:
a non-transitory memory device for storing computer readable program code; and
a processor device in communication with the non-transitory memory device, the processor device being operative with the computer readable program code to perform steps including
receiving projection data representing emissions detected from a subject,
formatting the projection data into energy-resolved data,
determining contribution coefficients of one or more components of the emissions based on the energy-resolved data, and
reconstructing an image of the subject using the contribution coefficients.

2. The image reconstruction system of claim 1 wherein the emissions have a continuous energy spectrum.

3. The image reconstruction system of claim 2 wherein the emissions are detected from bremsstrahlung radiation.

4. The image reconstruction system of claim 1 wherein the processor device is operative with the computer readable program code to format the projection data into the energy-resolved data by framing the projection data into multiple non-overlapping energy windows.

5. The image reconstruction system of claim 4 wherein at least one of the non-overlapping energy windows corresponds to an energy band of 5 keV.

6. The image reconstruction system of claim 4 wherein at least one of the non-overlapping energy windows is represented by an image size of 64 pixels by 64 pixels.

7. The image reconstruction system of claim 4 wherein the multiple non-overlapping energy windows comprise 20 or more non-overlapping energy windows.

8. The image reconstruction system of claim 1 wherein the processor device is operative with the computer readable program code to determine the contribution coefficients of the one or more components of the emissions by performing non-negative least squares regression (LSR) analysis on the energy-resolved data.

9. The image reconstruction system of claim 1 wherein the processor device is operative with the computer readable program code to determine the contribution coefficients of the one or more components of the emissions by performing Maximum-Likelihood Expectation-Maximization Algorithm (MLEM).

10. The image reconstruction system of claim 1 wherein the processor device is operative with the computer readable program code to reconstruct the image of the subject based on the contribution coefficients of primary and subject scatter components.

11. The image reconstruction system of claim 1 wherein the processor device is operative with the computer readable program code to further convert the contribution coefficients to weighted scaling factors representing primary and subject scatter components and reconstruct the image of the subject using the weighted scaling factors.

12. The image reconstruction system of claim 11 wherein the processor device is operative with the computer readable program code to convert the weighted scaling factors to higher spatial resolution weighted scaling factors.

13. A method, comprising:
receiving projection data representing emissions detected from a subject;
formatting the projection data into energy-resolved data;
determining contribution coefficients of one or more components of the emissions based on the energy-resolved data; and
reconstructing an image of the subject using the contribution coefficients.

14. The method of claim 13 wherein formatting the projection data into the energy-resolved data comprises framing the projection data into multiple non-overlapping energy windows.

15. The method of claim 14 wherein at least one of the non-overlapping energy windows corresponds to an energy band of 5 keV.

16. The method of claim 13 wherein determining the contribution coefficients comprises performing non-negative least squares regression (LSR) analysis on the energy-resolved data.

17. The method of claim 13 wherein determining the contribution coefficients comprises performing Maximum-Likelihood Expectation-Maximization Algorithm (MLEM).

18. The method of claim 13 wherein determining the contribution coefficients comprises performing a conjugate gradient method.

19. The method of claim 13 further comprises converting the contribution coefficients to weighted scaling factors representing primary and subject scatter components and reconstructing the image of the subject using the weighted scaling factors.

20. One or more non-transitory computer readable media embodying a program of instructions executable by machine to perform steps comprising:
receiving projection data representing emissions detected from a subject;
formatting the projection data into energy-resolved data;
determining contribution coefficients of one or more components of the emissions based on the energy-resolved data; and
reconstructing an image of the subject using the contribution coefficients.
